(19) United States
(12) Patent Application Publication
(10) Pub. No.: US 2015/0376152 A1
(43) Pub. Date: Dec. 31, 2015

(54) Process for Ultra Pure Chemical Production from Biobased Raw Starting Materials

(71) Applicant: METABOLIX, INC., Cambridge, MA (US)

(72) Inventors: Derek Samuelson, Somerville, MA (US); Oliver P. Peoples, Arlington, MA (US); Yossef Shabtai, Concord, MA (US); Johan Van Walsem, Acton, MA (US); Dirk Schweitzer, Cambridge, MA (US); Harvey H. Morgan, III, Cambridge, MA (US); Kevin A. Sparks, Scituate, MA (US)

(73) Assignee: Metabolix, Inc., Cambridge, MA (US)

(21) Appl. No.: 14/767,509

(22) PCT Filed: Feb. 12, 2014
(86) PCT No.: PCT/US2014/016122

§ 371 Date: Aug. 12, 2015

Related U.S. Application Data
(60) Provisional application No. 61/764,370, filed on Feb. 13, 2013.
(60) Provisional application No. 61/779,433, filed on Mar. 13, 2013.
(60) Provisional application No. 61/788,295, filed on Mar. 15, 2013.
(60) Provisional application No. 61/826,528, filed on May 23, 2013.
(60) Provisional application No. 61/833,659, filed on Jun. 11, 2013.
(60) Provisional application No. 61/839,385, filed on Jun. 26, 2013.

(51) Int. Cl. C07D 307/33; C07C 51/09; C07D 309/30; A61K 9/20; C07C 57/08; C07C 57/04

(52) U.S. Cl. CPC C07D 307/33; C07C 57/08; C07C 57/04; C07D 309/30; A61K 9/2013; C07C 51/09

(57) ABSTRACT

Processes and methods for making ultra-pure (>99.50% by weight), biobased crotonic acid, gamma-butyro lactone, acrylic acid and delta-valerolactone from renewable carbon resources are described herein.

### RELATED APPLICATIONS

This application claims the benefit of U.S. Provisional Application No. 61/764,370, filed on Feb. 13, 2013; U.S. Provisional Application No. 61/779,433, filed on Mar. 13, 2013; U.S. Provisional Application No. 61/788,295, filed on Mar. 15, 2013; U.S. Provisional Application No. 61/826,528, filed on May 23, 2013; U.S. Provisional Application No. 61/833,659, filed on Jun. 11, 2013; and U.S. Provisional Application No. 61/839,385, filed on Jun. 26, 2013. The entire teachings of the above applications are incorporated herein by reference.

### BACKGROUND OF THE INVENTION

The worldwide market for petroleum-based chemicals is currently estimated to be $3 trillion/year with the EU and US being the world's largest chemicals producers. However because of continued uncertainty with future petroleum supplies, increasing petroleum energy costs, and environmental concerns with petroleum manufacturing, the need to develop clean, energy efficient biorefinery processes to produce chemical and pharmaceutical products from low cost, renewable carbon resources is critical. There is also a need for very high purity chemical intermediates for use in pharmaceutical applications, in particular where larger doses, prolonged or continuous exposure is necessary. Much progress has already been made in developing processes which use renewable starting materials to produce chemicals. By the year 2015, the global renewable chemicals market is predicted to reach approximately $76 billion. However, there is still a need to develop higher yielding, environmentally neutral biorefinery processes which produce high purity, biobased chemicals and pharmaceuticals from renewable resource, including “just in time” chemical production.

### SUMMARY OF THE INVENTION

Integrated biorefinery processes for the production of ultra-high purity biobased chemicals from renewable carbon resources are described herein. The biobased chemicals include gamma-butyrolactone (GBL), crotonic acid, acrylic acid and delta-valerolactone. On advantage of the processes described herein is ability to convert from dried biomass for “just in time” chemical production e.g., chemical produced quickly on-site where needed avoiding such disadvantages as shipping problems of the chemical (like GBL) and storage.

In one aspect, a process is described for the production of biobased chemicals starting with genetically engineered microbes metabolizing glucose or any other renewable feedstock to produce polyhydroxyalkanoate polymers such as poly-3-hydroxybutyrate (P3HB), poly-4-hydroxybutyrate (P4HB), poly-3-hydroxypropionate (P3HP) or poly-5-hydroxyvalerate (P5HV) inside the microbial cells, (defined herein as a PHA biomass selected from P4HB biomass, P3HP biomass, P3HB biomass or P5HV biomass) followed by solvent extraction of an aqueous suspension of the biomass to remove the polymer, separation and isolation of the solvent and polymer extract fraction and finally combined heating/distillation under vacuum pressure of the solvent+polymer extract to produce a biobased chemical product. Optionally the heating/distillation can be carried out under atmospheric or a sequential combination of vacuum then atmospheric distillation. Optionally, after the isolation of the ultra-high purity biobased chemical, one or more additional distillations can be carried out to further remove trace impurities from the final biobased chemical product. Chemicals capable of being produced by this process include crotonic acid (from P3HB), gamma-butyrolactone (from P4HB), acrylic acid (from P3HP) and δ-valerolactone (from P5HV). The process is surprising in that it is capable of taking PHA biomass with an approximately 50% purity level, on a PHA weight basis, and in one step directly producing an ultra-pure (up to or greater than or 95.00%, 96.00%, 97.00%, 98.00%, 99.00%, 99.50% (e.g., 95.10 to 99.50%, such as 96.50%, 97.50%, 98.50%, 99.10%, 99.20%, 99.3%, 99.4%) by weight biobased chemical that is free from biomass or fermentation broth derived nitrogen-containing impurities such as acetamide, n-methyl pyrrolidone (NMP) and n-ethyl pyrrolidone (NEP) as well as other impurities such as fatty acids that negatively impact the biobased chemicals final color and odor properties. The product produced by the methods described herein has a greater than 95% weight without further processing to reach the purity. In other words, the purity of the product derived from the methods of the invention is between about 95% on a PHA weight basis without further processing such as distillation and the like to achieve the purity. In a certain aspect, the purity includes the product being odorless and with reduced color.

In a second aspect, a process for the production of ultra-pure, biobased chemical products is described, comprising, combining a genetically engineered biomass comprising a polyhydroxyalkanoate (PHA) and a solvent; mixing an aqueous suspension of the biomass and solvent together while optionally applying heat; followed by separation of the organic and aqueous phases of the biomass and solvent mixture; removing the solvent from the extracted PHA by heating; optionally adding an aqueous catalyst slurry or catalyst solution to the extracted PHA and converting the biomass comprising a polyhydroxyalkanoate to a biobased chemical product wherein the purity of the product is greater than 95% (e.g., 95%, 96%, 97%, 98%, 99% 99.2%. 99.3%. 99.4%99.50% by weight biobased chemical.

In a first embodiment of the second aspect, removing the solvent is accomplished by heating the organic phase containing solvent and polyhydroxyalkanoate, for example, under vacuum distillation. The heating can also be carried out under atmospheric distillation conditions.

In a second embodiment of the second aspect, wherein the method includes converting the extract from the biomass comprising poly-3-hydroxybutyrate (P3HB) by heating the solvent precipitate comprising P3HB under vacuum or atmospheric distillation to a higher temperature, wherein the remaining poly-3-hydroxybutyrate is converted to a crotonic acid product, wherein the weight percent crotonic acid in the product is greater than 99.50%, for example about 99.55%, about 99.60%, about 99.65%, about 99.70%, about 99.75%, about 99.80%, about 99.85%, about 99.90%, about 99.95%, about 99.96%, about 99.97%, about 99.98%, about 99.99% or about 100.00%.

In a third embodiment of the second aspect, wherein the method includes converting the extract from the biomass comprising poly-4-hydroxybutyrate (P4HB) by heating the solvent precipitate comprising P4HB under vacuum or atmospheric distillation to a higher temperature, wherein the remaining poly-4-hydroxybutyrate is converted to a GBL product, wherein the weight percent GBL in the product is greater than 99.50%, for example about 99.55%, about 99.60%, about 99.65%, about 99.70%, about 99.75%, about 99.80%, about 99.85%, about 99.90%, about 99.95%, about 99.96%, about 99.97%, about 99.98%, about 99.99% or about 100.00%.

In a fourth embodiment of the second aspect, the method includes converting the extract from the biomass comprising poly-3-hydroxypropionate (P3HP) by heating the solvent precipitate comprising P3HP under vacuum or atmospheric distillation to a higher temperature, wherein the remaining poly-3-hydroxypropionate is converted to an acrylic acid product, wherein the weight percent acrylic acid in the product is greater than 99.50%, for example about 99.55%, about 99.60%, about 99.65%, about 99.70%, about 99.75%, about 99.80%, about 99.85%, about 99.90%, about 99.95%, about 99.96%, about 99.97%, about 99.98% about 99.99% or about 100.00%.

In a fifth embodiment of the second aspect, the method includes converting the extract from the biomass comprising poly-5-hydroxyvalerate (P5HV) by heating the solvent precipitate comprising P5HV under vacuum or atmospheric distillation to a higher temperature, wherein the remaining poly-5-hydroxyvalerate is converted to a δ-valerolactone product, wherein the weight percent δ-valerolactone in the product is greater than 99.50%, for example about 99.55%, about 99.60%, about 99.65%, about 99.70%, about 99.75%, about 99.80%, about 99.85%, about 99.90%, about 99.95%, about 99.96%, about 99.97%, about 99.98% about 99.99% or about 100,00%.

In these aspects, the level of PHA in the starting biomass should be greater than 10% by weight of the total biomass. The advantages of this bioprocess are that it uses a renewable carbon source as the feedstock material; the genetically engineered microbe produces PHA in very high yield without adverse toxicity effects to the host cell (which could limit process efficiency) and when the PHA is subsequently removed from the biomass by solvent extraction followed by heating or other suitable methods, an ultra-high purity (e.g., greater than 99.50%, greater than 99.55%, greater than 99.60%, greater than 99.65%, greater than 99.70%, greater than 99.75%, greater than 99.80%, greater than 99.85%, greater than 99.90%, greater than 99.95% or greater than 99.99%), biobased chemical in high yield having almost no color (low APHA color value) and low detectable odor is produced. In any of the aspects or embodiments described above, the product has less than 0.50% side products (such as fatty acids, acetamide, NMP and NEP), for example, less than 0.40%, 0.30%, 0.20%, 0.10%, 0.05%, 0.04%, 0.03%, 0.02%, 0.01%, less than 0.005%, or a range between 0.001% and 0.50%. The reduction of the side products and the production of a 99.50% or higher product produced by the methods described herein are advantageous over other methods in the art. For example, it is advantageous that incorporating the steps of the method with optional heating generates such an advantageous product, without a significant increase in costs. The amount of side products are reduced and have a different composition than other methods. The reduction of side products helps to produce a visually colorless biochemical product having a low detectable odor.

Suitable solvents for extracting the PHA biomass includes those which have a strong affinity for the PHA polymers and can dissolve the PHA especially in the presence of water, have a boiling point different than water, have low miscibility with water and have a density different than water. Examples of preferred solvents include chlorinated alkanes, aromatics, lower ketones, cyclic ketones, alkyl carbonates, dialkyl ethers, lower alcohols and their esters, cyclic alcohols, fusel oil, lactides, lactones, acetates, diacetates, caproates, fumarates, butyrates, glycols, sulfoxides, formamides, dioxane and esters of renewable acids.

In certain aspects, a recombinant engineered PHA biomass from a host organism serves as a renewable source for converting P3HB, P4HB, P3HP and P5HV homopolymers to useful chemical and pharmaceutical intermediates such as crotonic acid, gamma-butyrolactone (GBL), acrylic acid and δ-valerolactone. In some embodiments, a source of the renewable feedstock for growing the PHA in the biomass is selected from glucose, fructose, sucrose, arabinose, maltose, lactose, xylose, fatty acids, vegetable oils, and biomass derived synthesis gas, natural gas or a combination of two or more of these. The produced PHA biomass whole broth which includes biomass, water, PHA and any residual dissolved nutrients and starting materials used to grow the biomass with PHA polymer, is then solvent extracted wherein the solvent with PHA polymer is separated from the biomass and the solvent with PHA is heated under vacuum, atmospheric or a sequential combination of the two in a distillation set up to produce ultra-high purity crotonic acid, gamma-butyrolactone (GBL), acrylic acid or delta-valerolactone. In other embodiments of any of the aspects, the PHA biomass is dried prior to combining with the solvent. In other embodiments the biomass is partially purified by centrifugation or filtration to wash away any water soluble compounds prior to solvent extraction of the PHA biomass or drying of the PHA biomass. In a further embodiment of any of the aspects, an aqueous catalyst slurry or catalyst solution is added to the PHA after the solvent has been removed but prior to thermolysis of the PHA. In certain embodiments of any of the aspects, the process further comprises recovering the ultra-high purity biobased chemical product. In certain embodiments of any of the aspects, the recovery is by condensation.

In some embodiments the biobased chemicals are further processed, derivatized or metathesized to other desired commodity and specialty products, for example: crotonic acid generated from P3HB can be converted via metathesis to acrylic acid, propene and 2-butene; gamma-hydroxybutyrate generated from P4HB can be converted to 1,4-butanediol (BDO), tetrahydrofuran (THF), N-methyl pyrrolidone (NMP), N-ethyl pyrrolidone (NEP), 2-pyrrolidinone, N-vinylpyrrolidone (NVP), polyvinylpyrrolidone (PVP), sodium oxybate and esters, including oligomeric esters of 4-hydroxybutyrate, and the like; acrylic acid generated from P3HP can be further derivatized to butyl acrylate, 1,3-propanediol or malonic acid.

In the methods described herein the host organism used to produce the biomass containing PHA has been genetically modified by introduction of genes and/or deletion of genes in a wild-type or genetically engineered production organism creating strains that synthesize the desired PHA from inexpensive renewable feedstock's. An exemplary pathway for production of P4HB for example is provided in FIG. 2 and it is understood that additional enzymatic changes that contribute to this pathway can also be introduced or suppressed for a desired production of P4HB.

In one aspect, the present invention provides a process for production of biobased crotonic acid, gamma-butyrolactone, acrylic acid or δ-valerolactone product. In certain embodiments, the product has 100% biobased carbon content (e.g., as determined based on ¹⁴C isotope analysis). The process includes combining a genetically engineered biomass broth, comprising polyhydroxyalkanoate, water and other fermentation nutrients, optionally pH adjusting the broth to 10.5-11.5 with lime; homogenizing or mixing the solvent with the pH adjusted biomass comprising PHA using known mixing techniques while also heating the mixture; separating the solvent and PHA phase from the biomass and other aqueous components; heating the solvent with PHA under vacuum or atmospheric distillation to remove the solvent; then heating to a higher temperature under vacuum or atmospheric distillation to thermolyze the PHA which converts the PHA to an ultra-high purity biobased chemical product. Alternatively, an aqueous slurry or solution of an acidic or basic catalyst can be added at a concentration of 0.1%-10% by weight PHA after removal of the solvent but prior to conversion of the PHA to an ultra-high purity biobased chemical.

In certain embodiments, a yield of biobased chemical product is about 85% by weight or greater based on one gram of a product per gram of the polyhydroxyalkanoate. The genetically engineered recombinant host produces a 3-hydroxybutyrate, 4-hydroxybutyrate, 3-hydroxypropionate or 5-hydroxyvalerate polymer. In certain embodiments, the weight percent of the biobased chemical in the final product is at least 99.5% or greater. In another embodiment, the biobased chemical contains no fatty acids and no nitrogen compounds such as acetamide, n-methyl pyrrolidone or n-ethyl pyrrolidone or these compounds are undetectable using analytical techniques such as gas chromatography-mass spectroscopy (GC-MS). In certain embodiments, the color of the biobased chemical as measured on the APHA scale is <20, <15, <10, <5 and the odor as measured by a human “sniff” test is acceptable (low detectable odor).

In another aspect, the genetically engineered biomass for use in the processes of the invention is from a recombinant host having a poly-3-hydroxybutyrate pathway or a poly-4-hydroxybutyrate pathway or a poly-3-hydroxypropionate pathway or a poly-5-hydroxyvalerate pathway.

In a certain aspect of the invention, the recombinant host is cultured with a renewable feedstock to produce a polyhydroxyalkanoate biomass, the produced biomass is then pH adjusted and extracted in the presence of an organic solvent utilizing a heater and vacuum distillation, atmospheric distillation or a sequential combination of the two to produce ultra-high purity, biobased chemical products, wherein a yield of biobased chemical product is at least 75%, 80%, 85, 90 or 95% by weight. In further aspects of the invention, the polyhydroxyalkanoate biomass prior to solvent extraction is dried or is dried and resuspended in water and combined with acids or bases that catalyze the conversion of the PHA to the biobased chemical. Alternatively, the liquid or solid catalyst, or an aqueous slurry or aqueous solution of the catalyst can be added after removal of solvent but prior to thermal conversion of the PHA to the biobased chemical. The amount of pure catalyst added is from 0.1%-10% by weight PHA. Preferred acidic and basic catalysts include compounds such as sulfuric acid, phosphoric acid, nitric acid, sodium bisulfate, sodium bicarbonate, sodium hydrogen sulfate, hydrochloric acid, trifluoroacetic acid, p-toluene sulphonic acid, methane sulphonic acid, zinc chloride, acetic acid, silica, titanium dioxide, alumina, calcium hydroxide (lime), sodium hydroxide, potassium hydroxide and potassium carbonate. Cation exchange resins can also be utilized such as DOWEX® HCR W2H. Most preferred are Lewis acid catalysts as they were found to generally have a low vapor pressure at the distillation pressures and therefore do not end up in the final biobased chemical product.

In certain embodiments, the source of the renewable feedstock is selected from glucose, fructose, sucrose, arabinose, maltose, lactose, xylose, fatty acids, vegetable oils, and biomass derived synthesis gas or a combination thereof.

The invention also pertains to biobased chemical products such as crotonic acid, gamma-butyrolactone, acrylic acid and δ-valerolactone as produced by the processes described herein. In certain aspects, the amount of biobased chemical in the final isolated product produced is 99.50% or greater than 99.50%. In a further aspect, the invention pertains to a polyhydroxyalkanoate biomass produced from renewable resources which is suitable as a feedstock for producing biobased chemical products, wherein the level of PHA in the biomass is greater than 50% by weight of the biomass.

In certain embodiments of the invention, the biomass host is bacteria, yeast, fungi, algae, cyanobacteria, or a mixture of any two or more thereof. The bacteria includes but is not limited to Escherichia coli, Alcaligenes eutrophus (renamed as Ralstonia eutropha), Bacillus spp., Alcaligenes latus, Azotobacter, Aeromonas, Comamonas, Pseudomonads), Pseudomonas, Ralstonia, Klebsiella), Synechococcus sp PCC7002, Synechococcus sp. PCC 7942, Synechocystis sp. PCC 6803, and Thermosynechococcus elongatus BP-I (cyanobacteria), Chlorobium tepidum (green sulfur bacteria), Chloroflexus auranticus (green non-sulfur bacteria), Chromatium tepidum and Chromatium vinosum (purple sulfur bacteria), Rhodospirillum rubrum, Rhodobacter capsulatus, and Rhodopseudomonas palustris, In other embodiments, the recombinant host is algae. The algae include but are not limited to Chlorella minutissima, Chlorella emersonii, Chlorella sorokiniana, Chlorella ellipsoidea, Chlorella sp., or Chlorella protothecoides.

In certain embodiments of the invention, the solvent is heated prior to mixing with the dry or aqueous fermentation biomass to 30° C., 40° C., 50° C., 60° C., 70° C., 80° C., 90° C., 100° C., 110° C., 120° C., 130° C., 140° C. or 150° C. After homogenization/mixing of the solvent and fermentation biomass, the mixture is sent to a distillation apparatus which is under atmospheric or vacuum conditions where it is first heated to 50° C., 60° C., 70° C., 80° C., 90° C., 100° C., 110 C, 120° C., 130° C. or 140° C., 150° C., 160° C. or 170° C. to remove the solvent. Once the solvent is removed, heating of the remaining solids is continued under vacuum or atmospheric conditions to temperatures of 50° C., 60° C., 70° C., 80° C., 90° C., 100° C., 110 C, 120° C., 130° C., 140° C., 150° C., 160° C., 170° C., 180° C., 185° C., 190° C., 195° C., 200° C., 205° C., 210° C., 215° C., 220° C., 230° C., 240° C. or 250° C. over a period of 1-4 hours in order to thermally degrade the PHA polymer into its monomer components thereby producing ultra-high purity biobased chemical vapors. The chemical vapors are then distilled and collected by condensation to form an ultra-high purity biobased chemical liquid (greater than 99.50% by weight monomer in the final product).

Additionally, the expended (residual) PHA reduced biomass is further utilized for energy development, for example as a fuel to generate process steam and/or heat. The ultra-high purity biobased chemical can also be further subjected to one or more distillations, ion exchange, activated carbon filtration or crystallization steps to further reduce trace level impurities.

### DETAILED DESCRIPTION OF THE INVENTION

A description of example embodiments of the invention follows.

The present invention provides processes and methods for the manufacture of ultra-high purity biobased chemicals from genetically engineered microbes producing poly-3-hydroxybutyrate polymer (P3HB biomass), poly-4-hydroxybutyrate polymer (P4HB biomass), poly-3-hydroxypriopionate polymer (P3HP biomass) or poly-5-hydroxyvalerate polymer (P5HV biomass). Addtionally P4HB, P3HP and P5HV are defined to also include their copolymers with 3-hydroxybutyrate monomer where the percent of 3-hydroxybutyrate in the copolymer is less than 20%, 15%, 10% preferably less than 5% of the monomers in the copolymer. In certain embodiments, the PHA biomass is produced by improved PHA production processes using recombinant hosts described herein. These recombinant hosts have been genetically engineered to increase the yield of PHA by manipulating (e.g., by inhibition and/or overexpression) certain genes in the PHA pathway to increase the yield of PHA in the biomass. The PHA biomass is produced in a fermentation process in which the genetically engineered microbe is fed a renewable substrate. Renewable substrates include fermentation feedstocks such as sugars, vegetable oils, fatty acids, methanol, ethanol, glycols, methane or synthesis gas produced from plant crop materials. The level of PHA produced in the biomass from the sugar substrate is greater than 10% (e.g., about 20%, about 30%, about 40%, about 50%, about 60%, about 70%, about 80%, about 90%) of the total dry weight of the biomass. At the end of fermentation, the PHA biomass broth is then extracted with a solvent and the solvent plus PHA polymer mixture is separated from the biomass and heated for example under vacuum or atmospheric conditions to first remove the solvent then secondly to thermally decompose (thermolyze) the PHA to its monomer units to produce an ultra-high purity biobased chemical. The conversion of the PHA to a biobased chemical product is accomplished by high efficiency, high yielding methodologies. As used herein, ultra-high purity or ultra-pure refers to biobased chemicals that comprise greater than 99.50% by weight of the chemical of interest including crotonic acid, gamma-butyrolactone, acrylic acid or delta-valerolactone or any combination of these as determined by GC-MS analysis or other appropriate analytical techniques capable of quantitating impurities to the parts per million (ppm) concentration in the final biobased chemical product. The ultra-pure biobased chemical so produced also has low APHA color and low detectable odor.

Described herein are alternative processes for manufacturing biobased chemicals based on using renewable carbon sources to produce a biobased PHA polymer (P3HB, P4HB, P3HP or P5HV) in a biomass that is then thermally converted to a biobased chemical (crotonic acid, GBL, acrylic acid or 6-valerolactone).

Biobased, biodegradable polymers such as polyhydroxyalkanoates (PHAs), are naturally produced in biomass systems, such as microbial biomass (e.g., bacteria including cyanobacteria, yeast, fungi), plant biomass, or algal biomass. Genetically-modified biomass systems have been developed which produce a wide variety of PHA polymers and copolymers in high yield (Lee (1996), Biotechnology & Bioengineering 49:1-14; Braunegg et al. (1998), J. Biotechnology 65:127-161; Madison, L. L. and Huisman, G. W. (1999), Metabolic Engineering of Poly-3-Hydroxyalkanoates; From DNA to Plastic, in: Microbiol. Mol. Biol. Rev. 63:21-53).

As described herein, solvent extraction of P4HB from biomass to create a solvent+P4HB phase followed by heating of solvent+P4HB phase under vacuum will also produce GBL by a similar thermal unzipping reaction of the polymer chain in solution. The thermal unzipping reaction surprisingly takes place at a much lower temperature (50° C.-190° C.) as compared to direct pyrolysis of the dry P4HB biomass (250° C. to 300° C.) which saves on production energy costs. When the heating is done under vacuum or atmospheric conditions and is combined with distillation, the process produces a much higher purity biobased chemical product that is free of nitrogen-containing compounds such as acetamide, n-methyl pyrrolidone (NMP) or n-ethyl pyrrolidone (NEP) and also has low APHA color and a low detectable odor. The advantages also include a more favorable economic and environmental alternative to the traditional petroleum-based processes for producing GBL. Similarly, the PHA polymers P3HB, P3HP and P5HV also “unzip” when heated near to their melting points to form respectively crotonic acid, acrylic acid and 8-valerolactone.

Recombinant Hosts with Metabolic Pathways for Producing PHA's

Genetic engineering of hosts (e.g., bacteria, fungi, algae, plants and the like) as production platforms for modified and new materials provides a sustainable solution for high value industrial applications for production of chemicals. Described herein are process methods of producing monomer components and other modified chemicals from a genetically modified recombinant polyhydroxyalkanoate (PHA) biomass. The processes described herein avoid toxic effects to the host organism by producing the biobased chemical post culture or post harvesting, are cost effective and highly efficient (e.g., use less energy to make), decrease greenhouse emissions, use renewable resources and can be further processed to produce high purity products in high yield.

As used herein, “PHA biomass” is intended to mean any genetically engineered biomass that includes a non-naturally occurring amount of polyhydroxyalkanoate polymer (PHA). The wild-type PHA biomass refers to the amount of PHA that an organism typically produces in nature. In certain embodiments, the biomass titer (g/L) of PHA has been increased when compared to the host without the overexpression or inhibition of one or more genes in the PHA pathway. In certain embodiments, the PHA titer is reported as a percent dry cell weight (% wdc) or as grams of PHA/Kg biomass. In some embodiments, a source of the PHA biomass is a plant crop, bacteria, yeast, fungi, algae, cyanobacteria, or a mixture of any two or more thereof.

Genetically engineered microbial PHA production systems with fast growing organisms such as Escherichia coli have been developed. Genetic engineering allows for the modification of wild-type microbes to improve the production of specific PHA copolymers or to introduce the capability to produce different PHA polymers by adding PHA biosynthetic enzymes having different substrate-specificity or even kinetic properties to the natural system. Examples of these types of systems are described in Steinbuchel & Valentin, FEMS Microbiol. Lett. 128:219-28 (1995). PCT Publication No. WO 1998/04713 describes methods for controlling the molecular weight using genetic engineering to control the level of the PHA synthase enzyme. Commercially useful strains, including Alcaligenes eutrophus (renamed as Ralstonia eutropha), Alcaligenes latus, Azotobacter vinlandii, and Pseudomonads, for producing PHAs are disclosed in Lee, Biotechnology & Bioengineering, 49:1-14 (1996) and Braunegg et al., (1998), J. Biotechnology 65: 127-161. In some embodiments, a source of the biomass includes the bacteria, E. coli. The E. coli may be one which has been genetically engineered to express or overexpress one or more PHAs. Exemplary strains, fermentation, media and feed conditions are described in U.S. Pat. Nos. 6,316,262; 6,323,010; 6,689,589; 7,081,357; 7,202,064 and 7,229,804.

Recombinant host containing the necessary genes that will encode the enzymatic pathway for the conversion of a carbon substance to PHA may be constructed using techniques known in the art.

For example, for the production of acrylic acid monomer, a genetically engineered host that produces P3HP is needed. For the production of poly-3-hyroxypropionate, recombinant hosts such as those described in U.S. Pat. Nos. 6,576,450, 6,316,262; 6,323,010; 6,689,589; 7,081,357; 7,202,064, and 7,229,804 can be used and are incorporated herein by reference. In general, if a host organism does not naturally produce PHA, genes for the P3PH pathway can be introduced. For example, to produce the 3HP polymers directly from carbohydrate feedstocks, host can be further engineered to express glycerol-3-phosphate dehydrogenase and glycerol-3-phosphatase. Such recombinant E. coli strains and methods for their construction are known in the art (U.S. Pat. No. 7,229,804 and Anton, D. “Biological production of 1,3-propanediol”, presented at United Engineering Foundation Metabolic Engineering II conference, Elmau, Germany, Oct. 27, 1998; PCT WO 1998/21339).

Recombinant hosts for producing polyhydroxyalkanoates (PHAs) comprising 5-hydroxyvalerate (5HV) monomers and methods of producing PHAs comprising 5HV monomers from renewable carbon substrates are described in International Application Pub. WO 2010/068953 and WO 2012/149162 and incorporated herein by reference. A recombinant host expressing genes encoding a polyhydroxyalkanoate (PHA) synthase and a 5-hydroxyvalerate-CoA (5HV-CoA) transferase or 5HV-CoA synthetase and at least one transgene encoding a heterologous enzyme involved in lysine catabolic pathways wherein the host produces a PHA polymer containing 5HV monomers when the organism is provided with a renewable carbon substrate selected from: lysine, starch, sucrose, glucose, lactose, fructose, xylose, maltose, arabinose or combinations thereof and the level of 5HV monomer produced is higher than in the absence of expression of the transgene(s) are provided. An exemplary host for production of poly 5-hydroxyvalerate expresses one or more genes encoding lysine 2-monooxygenase, 5-aminopentanamidase, 5-aminopetanoate transaminase, glutarate semialdehyde reductase, 5-hydroxy valerate CoA-transferase, and polyhydroxyalkanoate synthase to produce a PHA polymer containing 5HV monomers. Certain hosts have deletions or mutations in genes encoding glutarate semialdehyde dehydrogenase and/or lysine exporter encoding genes.

Also described are hosts with one or more of the genes encoding PHA synthase, 5HV-CoA transferase or 5HV-CoA synthetase is also expressed from a transgene to produce the poly-5-hydroxyvalerate polymers that can be used in the methods described herein.

An exemplary pathway for production of P4HB is provided in FIG. 2 and a more detailed description of the pathway and recombinant hosts that produce P4HB biomass is provided below. The pathway can be engineered to increase production of P4HB from carbon feed sources.

The weight percent PHA in wild-type biomass varies with respect to the source of the biomass. For microbial systems produced by a fermentation process from renewable resource-based feedstocks such as sugars, vegetable oils or glycerol, the amount of PHA in the wild-type biomass may be about 65 wt. %, 70 wt %, 75 wt %, 80 wt %, or 85 wt %, or more, of the total weight of the biomass

For example, in certain embodiments, the PHA is increased between about 20% to about 90% over the wild-type or between about 50% to about 80%. In other embodiments, the recombinant host produces at least about a 20% increase of P4HB over wild-type, at least about a 30% increase over wild-type, at least about a 40% increase over wild-type, at least about a 50% increase over wild-type, at least about a 60% increase over wild-type, at least about a 70% increase over wild-type, at least about a 75% increase over wild-type, at least about a 80% increase over wild-type or at least about a 90% increase over wild-type. In other embodiments, the P4HB is between about a 2 fold increase to about a 400 fold increase over the amount produced by the wild-type host. The amount of P4HB in the host or plant is determined by gas chromatography according to procedures described in Doi, Microbial Polyesters, John Wiley & Sons, p 24, 1990. In certain embodiments, a biomass titer of 100-120 g P4HB/Kg of biomass is achieved. In other embodiments, the amount of P4HB titer is presented as percent dry cell weight (% dcw).

### Culturing of Host to Produce PHA Biomass

In general, the recombinant host is cultured in a medium with a carbon source and other essential nutrients to produce the PHA biomass by fermentation techniques either in batches or continuously using methods known in the art. Additional additives can also be included, for example, antifoaming agents and the like for achieving desired growth conditions. Fermentation is particularly useful for large scale production. An exemplary method uses bioreactors for culturing and processing the fermentation broth to the desired product. Other techniques such as separation processes (centrifugation, filtration, sedimentation, spray drying) can be combined with fermentation for large scale and/or continuous production of PHA's.

As used herein, the term “feedstock” refers to a substance used as a carbon raw material in an industrial process. When used in reference to a culture of organisms such as microbial or algae organisms such as a fermentation process with cells, the term refers to the raw material used to supply a carbon or other energy source for the cells. Carbon sources useful for the production of crotonic acid, GBL, acrylic acid and δ-valerolactone include simple, inexpensive sources, for example, glucose, sucrose, lactose, fructose, xylose, maltose, arabinose and the like alone or in combination. In other embodiments, the feedstock is molasses or starch, fatty acids, vegetable oils, alcohols, glycols or a lignocelluloses material and the like. It is also possible to use organisms to produce the PHA biomass that grow on synthesis gas (CO₂, CO and hydrogen) produced from renewable biomass resources.

Introduction of P3HB, P4HB, P3HP and P5HV pathway genes allows for flexibility in utilizing readily available and inexpensive feedstocks. A “renewable” feedstock refers to a renewable energy source such as material derived from living organisms or their metabolic byproducts including material derived from biomass, often consisting of underutilized components like chaff or stover. Agricultural products specifically grown for use as renewable feedstocks include, for example, corn, soybeans, switchgrass and trees such as poplar, wheat, flaxseed and rapeseed, sugar cane and palm oil. As renewable sources of energy and raw materials, agricultural feedstocks based on crops are the ultimate replacement of declining oil reserves. Plants use solar energy and carbon dioxide fixation to make thousands of complex and functional biochemicals beyond the current capability of modern synthetic chemistry. These include fine and bulk chemicals, pharmaceuticals, nutraceuticals, flavanoids, vitamins, perfumes, polymers, resins, oils, food additives, bio-colorants, adhesives, solvents, and lubricants.

### Solvent Extraction of PHA Biomass

In general, following production (e.g., culturing) of the PHA comprising biomass, the PHA comprising biomass or whole broth is usually pH adjusted to a basic value (10.5-11.5) by adding lime or calcium hydroxide at 8-14 g/kg in order to stop biomass production. Whole broth is defined as the fermentation mixture containing biomass, PHA polymer, water, residual salts and other nutrients or media used to carry out the culturing. Once production is stopped, the whole broth is then solvent extracted to remove the PHA polymer from the biomass as described in U.S. Pat. Nos. 7,252,980, 7,713,720 and 7,567,173 incorporated herein by reference. Water may also be added to the whole broth prior to extracting with a solvent to reduce the overall percent solids of the broth in order to make it easier to process. Additionally, whole broth that has been previously dried where 95% of the water has been removed by drying methods known in the arts may be ground and then reconstituted with water prior to solvent extraction in order to make shipping of the PHA+biomass more convenient. In some embodiments, the biomass is initially dried before mixing with the solvent, for example at a temperature between about 100° C. and about 150° C. for an amount of time to reduce the water content of the biomass to below 1% by weight. The biomass can also be spray dried in order to reduce the amount of water to below 1% by weight. The solvent chosen for the extraction can be any which is capable of dissolving P3HB, P4HB, P3HP or P5HV polymers under dry or aqueous conditions to give a final percent solids content of 2-30% by weight of the solution, have a density difference of at least 0.1 kg/l as compared to water (1 kg/l), have a boiling point different than water of at least 20° C. and optionally have low miscibility with water. Suitable solvents include but are not limited to butyl acetate, isobutyl acetate, ethyl lactate, isoamyl acetate, benzyl acetate, 2-methoxy ethyl acetate, propyl propionate, butyl propionate, pentyl propionate, butyl butyrate, isobutyl isobutyrate, ethyl butyrate, ethyl valerate, methyl valerate, benzyl benzoate, methyl benzoate, dimethyl succinate, dimethyl glutarate, dimethyl adipate, isobutyl alcohol, 1-butanol, 2-methyl-1-butanol, 3-methyl-1 butanol, 1-pentanol, 3-pentanol, amyl alcohol, allyl alcohol, hexanol, heptanol, octanol, cyclohexanol, 2-ethylhexanol, tetrahydrofurfuryl alcohol, furfuryl alcohol, benzyl alcohol, fusel oil, 2-furaldehyde, methyl isobutyl ketone, methyl ethyl ketone, 2-butanone, 2-pentanone, 3-pentanone, 2-hexanone, acetone, toluene, xylene, benzene, super critical CO₂ or other gas, cyclohexanone, methylene chloride, tetrachloroethylene, trichloroethane, chloroform, gamma-butyrolactone, renewable acids and their esters derived from succinic, δ-valerolactone, methyl n-amyl ketone, 5-methyl-2-hexanone, ethyl benzene, 1,3-dimethoxybenzene, cumene, benzaldehyde, 1,2-propanediol, 1,2-diaminopropane, ethylene glycol diethyl ether, 1,2,3-trimethylbenzene, 1,2,4-trimethylbenzene, 1,3-dioxane, 1,4-dioxane, 1-nitropropane, acetic acid, acrylic acid, acetic anhydride, alpha-methylstyrene, acetophenone, toluene, ethylene glycol diacetate, dimethyl sulfoxide, dimethyl acetamide, dimethyl formamide and propylene carbonate, ethyl butyrate, propyl propionate, butyl propionate, tetrahydrofurfuryl acetate, methyl propionate, methyl n-valerate, ethyl valerate, 1-butanol, 2-methyl-1-butanol, 1-hexanol, ethylene glycol diacetate, acetophenone, 1,2-diaminopropane, acetic anhydride, dimethyl sulfoxide, propylene carbonate, tetrahydrofuran, 3-methyl-2-pentanone (butyl methyl ketone), 4-methyl-2-pentanone (methyl isobutyl ketone), 3-methyl-2-butanone (methyl isopropyl ketone), diisobutyl ketone, 2-methyl-3-heptanone (butyl isopropyl ketone), 2-heptanone, 3-heptanone (ethyl n-butyl ketone), 4-heptanone, 2-octanone (methyl n-hexyl ketone), 5-methyl-3-heptanone (ethyl amyl ketone), 5-methyl-2-hexanone, (methyl iso-amyl ketone), heptanone (pentyl methyl ketone), cyclo-pentanone, cyclo-hexanone, diethyl carbonate, diethylformamide, dimethyl carbonate, dimethyl succinate, dimethyl sulfoxide, dimethylformamide, 1,4-dioxane, ethyl acetate, ethylene glycol diacetate, methyl acetate, 1,1,2,2-tetrachloroethane, THF, 1,1,2-trichloroethane, 1,2,3-trichloropropane or any mixtures of these.

In a batch extraction process with whole broth, generally the volume ratio of whole broth to solvent is 1/1, 1/2, 1/3, 1/4 or 1/5. Once the solvent is added to the broth, the mixture is then heated to a temperature of 30° C., 40° C., 50° C., 60° C., 70° C., 80° C., 90° C., 100° C., 110° C., 120° C., 130° C., 140° C. or 150° C. or the solvent is preheated to these temperatures first then mixed with the whole broth. Heating of the solvent+broth mixture improves the contact efficiency of the solvent with the PHA and can help minimize the overall volume of solvent needed to extract the PHA from the biomass. The solvent and broth mixture is then homogenized using any mixing methods known in the arts such as high shear mixing, microfluidization, double stage homogenization, ultrasonic liquid processing or the like.

In the case where the solvent is immiscible with water, after mixing the solvent and broth, the broth+solvent solution is separated into two phases: either a higher density water/biomass phase and a lower density solvent/polymer phase or a higher density solvent/polymer phase and a lower density water/biomass phase. The solvent may also be miscible or partially miscible with water. Separation can be carried out by processes or equipment known in the arts such as centrifugation, single stage separatory funnel, spray columns, pulsed columns or settling tanks. When the solvent extraction efficiency and/or the separation efficiency for the process are high, the purity of the biobased chemicals formed can be greater than 99.99% by weight.

An additional embodiment uses a continuous process for both the extraction of the PHA polymer into an organic solvent and separation of the low and high density liquid phases. Equipment for carrying out continuous extraction includes but is not limited to extraction loops (with or without percolation filtering), multistage counter current extractors, aqueous two phase extractors and centrifuges. It has been found that ultra-high purity biobased chemicals are best produced in a continuous process using an extraction loop followed by centrifugation due to the fact the very high extraction and separation efficiency's additively contribute to achieving the highest purity chemicals (>99.9% by weight).

Removal of Organic Solvent Combined with Thermal Degradation of PHA

“Heating,” “pyrolysis”, “thermolysis” and “torrefying” as used herein refer to thermal degradation (e.g., decomposition) of the PHA polymer for conversion to a biobased chemical. In general, the thermal degradation of the PHA occurs at an elevated temperature, followed by distillation under vacuum or atmospheric conditions in a three stage process. In the first stage, the solvent+PHA solution is heated to a temperature such as 50° C., 60° C., 70° C., 80° C., 90° C., 100° C., 110° C., 120° C., 130° C., 140° C., 150° C., 160° C. or 170° C. in order to boil off the solvent and any residual water. The solvent that is removed in this way is then collected, dried to remove the water and recycled back into the extraction process (FIG. 1 shows a schematic of the process). In the second stage after the solvent is mostly removed, an acidic or basic catalyst can optionally be added to aid in the thermal breakdown of the PHA polymer. The catalyst is added as an aqueous solution or aqueous slurry where the weight percent catalyst is from 0.1%-10% by weight PHA. Preferred catalysts can be acidic or basic and include compounds such as sulfuric acid, phosphoric acid, nitric acid, sodium bisulfate, sodium bicarbonate, sodium hydrogen sulfate, hydrochloric acid, trifluoroacetic acid, p-toluene sulphonic acid, methane sulphonic acid, zinc chloride, acetic acid, silica, titanium dioxide, alumina, calcium hydroxide (lime), sodium hydroxide, potassium hydroxide and potassium carbonate. Cation exchange resins can also be utilized such as DOWEX® HCR W2H. Most preferred are Lewis acid catalysts as they are generally found to have the lowest vapor pressures at the distillation pressures utilized and therefore do not end up in the final biobased chemical product. The remaining solid PHA polymer is then thermolyzed under vacuum or atmospheric conditions by heating to 50° C., 60° C., 70° C., 80° C., 90° C., 100° C., 110° C., 120° C., 130° C., 140° C., 150° C., 160° C. or 170° C., 180° C., 185° C., 190° C., 195° C., 200° C., 205° C., 210° C., 215° C., 220° C., 230° C., 240° C. or 250° C. for a period of 1-4 hours generating a high purity biobased chemical vapor (crotonic acid, GBL, acrylic acid or δ-valerolactone) which is condensed and mildly refluxed until all of the polymer has been thermally degraded. The temperature at which thermolysis takes place is different for each PHA material and is dependent both on the chemical composition and structure of the PHA polymer. In the third stage, once the thermal degradation is complete, a vacuum is slowly applied and the refluxing liquid is sent to a distillation column. Alternatively the system can be kept under atomspheric conditions while distillation proceeds. The ultra-high purity biobased liquid is then collected by condensing into a receiving vessel.

In the case when GBL or acrylic acid or δ-valerolactone liquid itself is used as the extraction solvent, only a single stage heating of the solvent+P4HB solution is carried out at a temperature of at least 205° C. The weight percent GBL, crotonic acid, acrylic acid or delta-valerolactone in the vapor or condensed liquid phase is greater than 99.50%, for example about 99.55%, about 99.60%, about 99.65%, about 99.70%, about 99.75%, about 99.80%, about 99.85%, about 99.90%, about 99.95%, about 99.96%, about 99.97%, about 99.98%, about 99.99% or about 100.00%. The biobased chemicals also have undetectable concentrations of nitrogen-containing compounds such as fatty acids, sulfur compounds, acetamide, NMP or NEP as measured by analytical techniques such as GC-MS. This is due to the fact that these compounds are generated during thermolysis of biomass cells. Removal of the biomass cells prior to thermolysis of the polymer therefore eliminates the source of these impurities in the final biobased chemical product. The APHA color value of the ultra-high purity biobased chemical liquids collected can be for example 20, 15, 10, 5 or <5.

The detectable odor by humans of the ultra-high purity biobased chemical produced as described herein has also been found to be very low. Low odor is particularly important in pharmaceutical applications where the biobased chemical is ingested by humans eg. for biobased GBL used in sodium oxybate production for the treatment of narcolepsy. The Odor Detection Threshold (ODT) is the lowest concentration (in water or air) for an odor compound than can be perceived by the human sense of smell and is dependent on among other things the compounds molecular shape, polarity, surface charge and molecular mass. There are published tables of ODT values available for many organic compounds. These have been generated traditionally through extensive testing with human subjects using controlled laboratory settings. Generally the lower the ODT for a compound, the easier it is for humans to detect this compound in air, water or other matrices. In the last ten years, equipment has been developed to mimic the human olfactory sense for measuring odor or odor patterns in products. One such instrument is the ZNOSE® manufactured by the Electronic Sensor Technology Company. This instrument uses gas chromatography coupled with a surface acoustic wave (SAW) quartz detector connected to a pump to sample or “sniff” materials to identify and quantitate volatile hydrocarbon based odor bodies. Because the instrument utilizes an array of detectors, odor maps can be generated which are used to quickly assess the purity of raw materials for processes for example. The materials tested with this instrument include plastics, food and beverages, environmental samples, cosmetics, explosives, biologicals, pharmaceuticals and fragrances.

In certain embodiments, the PHA biomass is dried prior to solvent extraction for example by spray drying. Alternatively, in other embodiments, water is removed during the solvent extraction of the PHA biomass. Drying reduces the water content of the biomass. In certain embodiments, the biomass is dried at a temperature of between about 100° C. to about 350° C., for example, between about 200° C. and about 275° C. In some embodiments, the dried 4PHB biomass has a water content of 5 wt. % or less. The remainder of any water present in the solvent and PHA polymer solution is subsequently removed during distillation.

In certain embodiments, “recovering” the gamma-butyrolactone, crotonic, acrylic acid or δ-valerolactone vapor includes condensing the vapor. As used herein, the term “recovering” as it applies to the vapor means to isolate it from the solid PHA polymer, for example including but not limited to: recovering by condensation, separation methodologies, such as the use of membranes, gas (e.g., vapor) phase separation, such as distillation, and the like. Thus, the recovering may be accomplished via a condensation mechanism that captures the monomer component vapor, condenses the monomer component vapor to a liquid form and transfers it away from the solid PHA polymer.

As a non-limiting example, the condensing of the biobased chemical vapor may be described as follows: the incoming gas/vapor stream from the distillation chamber enters an interchanger, where the gas/vapor stream may be pre-cooled. The gas/vapor stream then passes through a chiller where the temperature of the gas/vapor stream is lowered to that required to condense the designated vapors from the gas by indirect contact with a refrigerant. The gas and condensed vapors flow from the chiller into a separator, where the condensed vapors are collected in the bottom. The gas, free of the vapors, flows from the separator, passes through the Interchanger and exits the unit. The recovered liquids flow, or are pumped, from the bottom of the separator to storage.

After recovering the ultra-high purity biobased liquid, it can be further processed or purified by techniques known in the art such as distillation (vacuum, reactive), ion exchange, activated carbon filtration, liquid-liquid extraction, crystallization or any combination of these.

The processes described herein provide a yield of biobased chemicals expressed as a percent yield, for example, when grown from glucose as a carbon source, the yield is up to 95% based gram chemical recovered per gram PHA contained in the biomass fed to the process (times 100%). In other embodiments, the percent yield is in a range between about 40% and about 95%, for example between about 50% and about 70%, or between about 60% and 70%. In other embodiment, the yield is about 75%, about 70%, about 65%, about 60%, about 55%, about 50%, about 45% or about 40%.

As used herein, crotonic acid refers to the compounds with the following chemical structures:

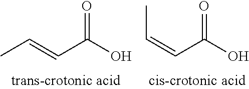

The term “crotonic acid product” refers to a product that contains at least about 99 up to 100 weight percent crotonic acid. For example, in a certain embodiment, the crotonic acid product may contain 99% by weight crotonic acid and 1% by weight side products. In some embodiments, the amount of crotonic acid in the crotonic acid product is greater than 99.50%, for example about 99.55%, about 99.60%, about 99.65%, about 99.70%, about 99.75%, about 99.80%, about 99.85%, about 99.90%, about 99.95%, about 99.96%, about 99.97%, about 99.98%, about 99.99% or about 100% by weight. In certain embodiments, the weight percent of crotonic acid product produced by the processes described herein is 99.99% or greater than 99.99%. The crotonic acid product contains undetectable concentrations of water, fatty acids, acetamide, NMP and NEP as measured by GC-MS.

As used herein, “gamma-butyrolactone” or GBL refers to the compound with the following chemical structure:

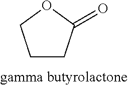

The term “gamma-butyrolactone product” refers to a product that contains at least about 99 up to 100 weight percent gamma-butyrolactone. For example, in a certain embodiment, the gamma-butyrolactone product may contain 99% by weight gamma-butyrolactone and 1% by weight side products. In some embodiments, the amount of gamma-butyrolactone in the gamma-butyrolactone product is greater than 99.50%, for example about 99.55%, about 99.60%, about 99.65%, about 99.70%, about 99.75%, about 99.80%, about 99.85%, about 99.90%, about 99.95%, about 99.96%, about 99.97%, about 99.98%, about 99.99%, or about 100% by weight. In certain embodiments, the weight percent of gamma-butyrolactone product produced by the processes described herein is 99.99% or greater than 99.99%. The GBL product contains undetectable concentrations of of water, fatty acids, acetamide, NMP and NEP as measured by GC-MS.

As used herein, “acrylic acid” refers to the compound with the following chemical structure:

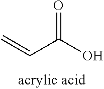

The term “acrylic acid product” refers to a product that contains at least about 99 up to 100 weight percent acrylic acid. For example, in a certain embodiment, the acrylic acid product may contain 99% by weight acrylic acid and 1% by weight side products. In some embodiments, the amount of acrylic acid in the acrylic acid product is greater than 99.50%, for example about 99.55%, about 99.60%, about 99.65%, about 99.70%, about 99.75%, about 99.80%, about 99.85%, about 99.90%, about 99.95%, about 99.96%, about 99.97%, about 99.98%, about 99.99%, or about 100% by weight. In certain embodiments, the weight percent of acrylic acid product produced by the processes described herein is 99.99% or greater than 99.99%. The acrylic acid product contains undetectable concentrations of water, fatty acids, acetamide, NMP and NEP as measured by GC-MS.

As used herein, “δ-valerolactone” refers to the compound with the following chemical structure:

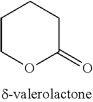

The term “δ-valerolactone product” refers to a product that contains at least about 99 up to 100 weight percent δ-valerolactone. For example, in a certain embodiment, the acrylic acid product may contain 99% by weight δ-valerolactone and 1% by weight side products. In some embodiments, the amount of δ-valerolactone in the δ-valerolactone product is greater than 99.50%, for example about 99.55%, about 99.60%, about 99.65%, about 99.70%, about 99.75%, about 99.80%, about 99.85%, about 99.90%, about 99.95%, about 99.96%, about 99.97%, about 99.98%, about 99.99%, or about 100% by weight In certain embodiments, the weight percent of δ-valerolactone product produced by the processes described herein is 99.99% or greater than 99.99%. The δ-valerolactone product contains undetectable concentrations of water, fatty acids, acetamide, NMP and NEP as measured by GC-MS.

In other embodiments, the biobased chemical products can be further purified if needed by additional methods known in the art, for example, by additional distillation steps including reactive distillation (e.g., the chemical product is acidified first to oxidize certain components (e.g., for ease of separation)) and then distilled followed by treatment with activated carbon for removal of color and/or odor bodies, vacuum distillation, extractive distillation or similar methods that would result in further purifying the biobased chemical to increase the purity and yield. Combinations of these treatments can also be utilized.

In certain embodiments, the biobased chemical products are further chemically modified and/or substituted to produce other products and derivatives. For example crotonic acid can be converted to acrylic acid, propene and 2-butene via metathesis reactions or to butanol, 1,4-butanediol or maleic anhydride via hydrogenation/oxidation reactions; GBL can be converted to succinic acid, 1,4-butanediamide, succinonitrile, succinamide, N-vinyl-2-pyrrolidone (NVP), 2-pyrrolidone (2-Py), N-methyl-2-pyrrolidone (NMP), tetrahydrofuran (THF), 1,4-butanediol (BDO) and sodium oxybate; acrylic acid can be converted to its butyl acrylate ester. Methods and reactions for production of these derivatives are readily known by one skilled in the art. For any of the catalytic conversion of the biobased chemicals to be successful, it is important to minimize impurities which can contribute to poisoning of the catalysts. Nitrogen-containing compounds such as acetamide, NMP, NEP or 2-pyrrolidone are typical impurities that are often generated from heating biomass which can lead to catalyst poisoning. The processes described herein produces biobased chemicals which eliminate these nitrogen compounds as well as other impurities which can poison catalysts thereby limiting their usefulness as starting materials and contribute negatively to other properties such as color.

As used herein, the term “residual biomass” refers to the biomass+nutrients+starting materials that remain after the P4HB polymer has been extracted out with organic solvent and separated from the solvent+P4HB polymer solution. The residual biomass obtained may then be converted via torrefaction or other heating methods to a useable, fuel, thereby reducing the waste from PHA production and potentially gaining additional valuable commodity chemicals from typical torrefaction processes. The torrefaction is conducted at a temperature that is sufficient to densify the residual biomass. In certain embodiments, processes described herein are integrated with a torrefaction process where the residual biomass is to be thermally treated once the PHA polymer is extracted to provide a fuel material. Fuel materials produced by this process are used for direct combustion or further treated to produce pyrolysis liquids or syngas. Overall, the process has the added advantage that the residual biomass is converted to a higher value fuel which can then be used for the production of electricity and steam to provide energy for the process thereby eliminating the need for waste treatment.

A “carbon footprint” is a measure of the impact the processes have on the environment, and in particular climate change. It relates to the amount of greenhouse gases produced.

In certain embodiments, it may be desirable to label the constituents of the biomass. For example, it may be useful to deliberately label with an isotope of carbon (e.g., ¹³C) to facilitate structure determination or for other means. This is achieved by growing microorganisms genetically engineered to express the constituents, e.g., polymers, but instead of the usual media, the bacteria are grown on a growth medium with ¹³C-containing carbon source, such as glucose, pyruvic acid, etc. In this way polymers can be produced that are labeled with ¹³C uniformly, partially, or at specific sites. Additionally, labeling allows the exact percentage in bioplastics that came from renewable sources (e.g., plant derivatives) can be known via ASTM D6866-an industrial application of radiocarbon dating. ASTM D6866 measures the Carbon 14 content of biobased materials; and since fossil-based materials no longer have Carbon 14, ASTM D6866 can effectively dispel inaccurate claims of biobased content

### EXAMPLES

The present technology is further illustrated by the following examples, which should not be construed as limiting in any way.

### Experimental Methods

### Measurement of Biobased Chemical Purity by Gas Chromatography-Mass Spectroscopy (GC-MS)

To determine the weight percent impurities in biobased liquid chemical samples an Agilent Model 7890 A gas chromatograph equipped with an MSD 5790 mass spectral detector, autosampler and J & W HP-5MS, 30 meter, capillary column was used. To a 1 ml sample of biobased chemical having unknown purity was added 3 μl of 2-pentanone (Ultrapure grade, >99%, Sigma Aldrich) as an internal standard. The solution was placed in a GC vial and place in the autosampler. The GC oven was programmed to hold at 70° C. for 3 minutes, then ramp @20° C./min to 250° C. and hold for 5 minutes then ramp to 270° C. and hold for 1 minute. Total run time was 19 minutes. The MSD detector was programmed to shut off during elution of the chemical at approximately 3.5-6 minutes. Once the GC and MSD were programmed, a 10 μl sample of the liquid biobased chemical sample with 2-pentanone was injected onto the GC column. After the GC program was completed, the GC peaks relating to the compounds present as impurities as well the 2-pentanone were integrated and the weight percent purity of the biobased chemical was calculated from the data. The detection limits for the GC-MS method for measuring the impurities in the biobased chemical were on the order of 200 ppm.

These examples describe a number of biotechnology tools and methods for the construction of strains that generate a product of interest. Suitable host strains, the potential source and a list of recombinant genes used in these examples, suitable extrachromosomal vectors, suitable strategies and regulatory elements to control recombinant gene expression, and a selection of construction techniques to overexpress genes in or inactivate genes from host organisms are described. These biotechnology tools and methods are well known to those skilled in the art.

### Example 1

### Production of Biobased GBL from Pyrolysis of Dried Whole Fermentation Broth

This is a comparative example describing a process for producing biobased GBL from pyrolysis of whole dried fermentation broth containing biomass with P4HB polymer prepared via fermentation using an Ecoli strain engineered to maximize the production of poly-4-hydroxybutyrate polymer as published in WO 2011/100601.

Biomass containing poly-4-hydroxybutyrate (poly-4HB) was produced in a 20 L New Brunswick Scientific fermentor (BioFlo 4500) using a genetically modified E. coli strain specifically designed for high yield production ofpoly-4HB from glucose syrup as a carbon feed source. Examples of the E. coli strains, fermentation conditions, media and feed conditions are described in U.S. Pat. Nos. 6,316,262; 6,689,589; 7,081,357; and 7,229,804. The E. coli strain generated a fermentation broth which had a PHA titer of approximately 100-120 g of PHA/kg of broth. After fermentation, the broth was washed with DI water by adding an equal volume of water, mixing for 2 minutes, centrifuging and decanting the water. Next, the washed broth was mixed with lime (Ca(OH)₂ standard hydrated lime 98%, Mississippi Lime) targeting 4% by wt dry solids. The mixture was then dried in a rotating drum dryer at 125-130° C. to a constant weight. Moisture levels in the dried biomass were approximately 1-2% by weight. The final wt % calcium ion in the dried broth+P4HB was measured by Ion Chromatography to be 1.9% (3.5% by wt. Ca(OH)₂).

Pyrolysis of the dried broth+P4HB+Ca(OH)₂ was carried out using a rotating, four inch diameter quartz glass kiln suspended within a clamshell tube furnace. At the start of the process, a weighed sample of dried broth+P4HB+Ca(OH)₂ was placed inside of the glass kiln and a nitrogen purge flow established. The furnace rotation and heat up was then started. As the temperature of the furnace reached its set point value of 250° C., gases generated by the broth+P4HB+Ca(OH)₂ sample would be swept out of the kiln by the nitrogen purge and enter a series of glass condensers or chilled traps. The condensers consisted of a vertical, cooled glass condenser tower with a condensate collection bulb located at its base. A glycol/water mixture held at 0° C. was circulated through all of the glass condensers. The cooled gases that exited the top of the first condenser were directed downward through a second condenser and through a second condensate collection bulb before being bubbled through a glass impinger filled with deionized water.

For the pyrolysis experiment, 292 g of dried broth+P4HB+Ca(OH)₂ was first loaded into the quartz kiln at room temperature. The total weight of P4HB biomass was estimated to be 281.4 g based on Ca(OH)₂ loading. The wt % P4HB in the mixture was also measured to be 66.7% (see Doi, Microbial Polyesters, John Wiley and Sons, p 23, 1990) based on the dry solids which made the mass of P4HB in the kiln equal to 195 g. The system was then sealed up and a nitrogen purge of approximately 1500 ml/min was established. Power was applied to the furnace and the dried broth+P4HB+Ca(OH)₂ was heated up to the target pyrolysis temperature of 250° C. During pyrolysis, the products of thermal degradation of biomass+P4HB, GBL, were collected in the condensate traps below the cooled condensers. Water could be seen to collect initially in each of the collection bulbs. The majority of the liquefied product (>95%) was collected in the first glass collection bulb. Total pyrolysis run time was approximately 60 minutes. The weight of the remaining biomass after pyrolysis was measured to be 11.9 g.

After the completion of pyrolysis, the liquids from the condensers were collected and analyzed by GC-MS. The % Weight GBL in the condensation mixture collected was determined to be approximately 85%. The major impurities identified included water, GBL dimer, fatty acids, nitrogen and sulfur-containing compounds. The sample was further processed using two additional distillation steps which resulted in a final percent weight GBL of approximately 99%. The biobased content as measured using ASTM D6866 was determined to be 100%.

### Example 2

### Production of Biobased GBL from Solvent Extracted Whole Fermentation Broth

This example describes a process for producing biobased GBL from whole fermentation broth which contained biomass with P4HB polymer, water and any dissolved residual nutrients or starting mateirals used to grow the PHA polymer prepared via fermentation using an Ecoli strain engineered to maximize the production of poly-4-hydroxybutyrate polymer whereby the P4HB polymer is first solvent extracted and then the solution thermolyzed under vacuum.

Aqueous fermentation broth consisting of E. coli microbial biomass containing poly-4-hydroxybutyrate (P4HB) polymer was prepared from glucose as described in International Patent Pub. WO2011/100601 to Metabolix. The PHA titer for the broth was measured to be 175.8 g P4HB/kg of broth with a washed dry cell content (WDC) of 23.4%. The fermentation broth was pH adjusted to 11.5 by the addition of approximately 8.5 g of lime/kg broth (Ca(OH)₂, Sigma Aldrich). 800 g of the pH 11.5 adjusted broth then was added to a 2 liter centrifuge flask and combined with 776 ml of reagent grade 2-pentanone (Sigma Aldrich, 90% pure containing up to 10% MIBK) that had been heated to 60-65° C. prior to mixing. The 2 liter centrifuge flask was placed in a water bath set at 60-65° C. for 1 hour and the contents homogenized using a mixer every 5-10 minutes. After 1 hour, the centrifuge flask was removed from the water bath, placed in a heated centrifuge and spun down at 2200 rpm for 2 minutes. After centrifugation, the top liquid layer containing the P4HB polymer and 2-pentanone was decanted into a glass 1 liter distillation pot. Total weight of the liquid was 651.3 g having 17.9% by weight solids. The distillation pot was then placed into a heating mantle and connected to a vacuum distillation set up. The heating mantle was turned on and adjusted to hold at a temperature of 180-185° C. The vacuum was also turned on and set to 700 mmHg. In the first stage, the solvents were boiled over into a receiving flask @102° C. for the 2-pentanone and 117.5° C. for the MIBK. The vacuum and heating mantle were shut off and the system vented to the atmosphere in order to remove and weigh the solvent collected. Total yield for the solvent recovery was measured to be 84.9%. The system was reconnected together with a clean collection flask and the vacuum and heating mantle started again. The vacuum pressure was measured to be 712 mmHg. During the second stage heating, the P4HB polymer remaining in the distillation pot melted and then thermally degraded to produce GBL vapor which was refluxed back into the pot. In the third stage, the GBL liquid was distilled, condensed and collected into a clean receiving flask. When the distillation pot became completely dry, the vacuum and heating mantle were shut off the and the GBL liquid allowed to cool to room temperature. Based on the weight of the GBL liquid and % solids in the extraction solvent, the total yield was calculated to be 76.3% (g GBL/g P4HB). The purity of the liquid GBL collected was measured by GC-MS to be 99.82% with total impurities of about 810 ppm. The impurities did not contain any biomass-generated nitrogen-containing compounds such acetamide, NMP or NEP as was typically found when the dry biomass was pyrolyzed directly without the solvent removal step. The main impurities were found to consist of residual extraction solvents 2-pentanone and MIBK. The biobased content of the GBL recovered as measured by ASTM 6866 was measured to be 100%. This was compared to the GC-MS analysis of commercially available GBL (Sigma Aldrich, cat#B103608) which showed the % Weight GBL to be 99.85% and a biobased content of 0%.

### Example 3

### Generation of Biobased GBL from Continuously Extracted Whole or Spray Dried Broth

In this example, ultra high purity GBL is produced from a poly-4-hydroxybutyrate/solvent solution that was prepared by extracting whole or water reconstituted spray dried broth using a continuous extraction/separation process.

The process involves the continuous mixing of aqueous fermentation broth with a heated solvent stream. Typical solvents to extract the poly-4-hydroxybutyrate from the whole broth could include 2-pentanone, 3-pentanone, cyclohexanone, methyl ethyl ketone or acetone. The mass ratio of the solvent to broth used within the process is typically maintained at a value of less than 1.0. During the process, a pair of control pumps are used to maintain targeted flows of broth and solvent feeding the process. Heated solvent is blended directly with the unheated broth in a mixing loop (extraction loop) utilizing an oversized centrifugal pump to circulate the mixture around the mixing loop. The volume of the loop is designed to provide approximately 5-10 minutes of continuous mixing prior to the downstream separation. The extraction loop behaves very much like a fixed volume continuous stirred tank reactor (CSTR). The loop additionally contains a pair of in-line static mixers and direct controlled steam injection to maintain the temperature of the mixture at a fixed targeted extraction temperature of 55° C. to 80° C. This type of set up provides intimate mixing of the broth with the solvent thereby creating very high polymer extraction efficiencies.

The mixture leaving the extraction loop is then sent through a high shear mixing pump and then on into a decanting type extraction centrifuge. The decanting centrifuge performs the primary separation of the heavier aqueous stream (spent broth) from the lighter polymer containing solvent stream assuming that the density of the solvent being utilized is less than that of water. Upon separation, the spent broth is directed to waste processing where the solvent is recovered and recycled to the front of the process. The polymer containing solvent stream is then mixed with additional water just upstream of a second, higher speed, disk stack polishing centrifuge for final separation. The heavier aqueous waste stream is similarly redirected to waste processing for solvent recovery. The combination of the two centrifuges (decanting and polishing) provides a very high separation efficiency to remove the biomass or spent cells from the polymer thereby giving a high purity polymer/solvent solution.

The resulting high purity solvent/polymer stream is then sent to a semi-continuous thermolysis reactor where the solvent is stripped from the polymer and accumulated within the reactor. Upon the filling of the reactor with a sufficient volume of polymer solution, the solvent recovery process is shut down. The solvent stripping portion of the thermolysis step is then completed by heating the solution up to 150-180° C. After the solvent is removed, the polymer then undergoes thermal degradation resulting in the generation of the targeted biobased monomer, gamma-butyrolactone (GBL). Ultimately, this material is vacuum stripped from the reactor through a packed column with reflux for enhanced purification resulting in a high purity (>99.9% by weight) condensate stream. A block diagram of the entire process is illustrated in FIG. 3. The biobased content of the GBL as measured by ASTM D6866 was 100%.

### Example 4

### Production of Biobased GBL from Purified P4HB

In this example biomass containing P4HB is produced in a fermentation process using glucose as the sole carbon feed source as described in International Patent Pub. WO2011/100601 to Metabolix. Following the fermentation, the P4HB is then extracted from the biomass and purified. Suitable methods for purifying P4HB from biomass are described in for example U.S. Pat. No. 6,610,764 to Tepha and Metabolix and U.S. Pat. Nos. 7,981,642 and 7,576,173 to Metabolix Inc. Purified P4HB polymer is subjected to the thermolysis procedure essentially under the same conditions as described in Example 1 and GBL is produced. GBL produced using this approach should have a purity of >99.90% and contain no nitrogen compounds such as acetamide, NMP or NEP. The biobased content of the GBL would be 99%-100% when tested according to the standard ASTM-D6866-11 testing protocol.

### Example 5

### Generation of Biobased Crotonic Acid from P3HB Biomass

This example describes a process for producing biobased crotonic acid from whole fermentation broth containing biomass with poly-3-hydroxybutyrate (P3HB). Alternatively in this example, plant biomass such as tobacco, switchgrass, sugarcane or camelina seeds containing P3HB polymer could be used as the starting material for generating crotonic acid (see US Pub. 2009/0271889, WO 2011/034946 and WO 2010/102217).

Aqueous fermentation broth consisting of E. coli microbial biomass containing poly-3-hydroxybutyrate polymer is prepared from glucose using procedures described in U.S. Pat. Nos. 6,316,262; 6,323,010; 6,689,589; 7,081,357; 7,202,064 and 7,229,804. The PHA titer for the broth would be approximately 150-180 g P3HB/kg of broth. The fermentation broth is pH adjusted to 11.5 by the addition of 8.5 g of lime/kg broth (Ca(OH)₂, Sigma Aldrich). 800 g of the pH 11.5 adjusted broth is then added to a 2 liter centrifuge flask and combined with 700 ml of reagent grade cyclohexanone (Sigma Aldrich) that is heated to 60-65° C. prior to mixing. The 2 liter centrifuge flask is placed in a water bath set at 60-65° C. for 1 hour and the contents are homogenized using a mixer applied every 5-10 minutes. After 1 hour, the centrifuge flask is removed from the water bath, placed in a heated centrifuge and spun down at 2200 rpm for 2 minutes. After centrifugation, the top liquid layer containing the P3HB polymer and cyclohexanone is decanted into a glass 1 liter distillation pot. The distillation pot is then placed into a heating mantle and connected to a vacuum distillation set up. The mantle is heated to 180-185° C. while a vacuum is applied and set to 700 mmHg. Initially the solvent is boiled over and condenses into a receiving flask @155° C. During the second stage heating, the P3HB polymer remaining in the distillation pot melts and then is thermally degraded to produce crotonic acid vapor. The crotonic acid vapor is refluxed until all of the P3HB polymer is degraded. In the third stage, the refluxing is stopped and the crotonic acid vapor is sent through the distillation column under atmospheric conditions, condensed and collected in the receiving flask. When the distillation pot is dry, the vacuum and heating mantle are shut off and the crotonic acid liquid is allowed to cool to room temperature. Total yield of crotonic acid recovered would be 75-80% (g crotonic acid/g P3HB×100%). The purity of the crotonic acid is expected to be >99.5% with a biobased content of 99-100%.

### Example 6

### Generation of Acrylic Acid from P3HP Biomass

This example describes a process for producing biobased acrylic acid from whole fermentation broth containing biomass with poly-3-hydroxypropionate (P3HP).

Aqueous fermentation broth consisting of E. coli microbial biomass containing poly-3-hydroxypropionate polymer was prepared in a 20 L fermentor from glucose using procedures described in U.S. Pat. Nos. 6,329,183 and 8,114,643. The PHA content of the biomass was approximately 50% by weight dry broth or 40-50 g P3HP/kg wet broth. 1000 ml of the aqueous broth was heated to 50-55° C. then added to a 2 liter centrifuge flask and combined with 800 ml of reagent grade 2-butanone (Sigma Aldrich) that was also heated to 50-55° C. prior to mixing with the broth. Alternatively, chloroform, methylene chloride or 2-pentanone solvents could also be utilized to extract the P3HP from the biomass. The 2 liter centrifuge flask was placed in a water bath set at 70° C. for 30 minutes and the contents were homogenized using a mixer applied every 5-10 minutes. After 30 minutes, a 500 ml portion of the solvent/broth mixture was removed, placed in a heated centrifuge and spun down at 2200 rpm for 2 minutes. After centrifugation, the top liquid layer containing the P3HP polymer and 2-butanone was decanted into a glass 1 liter distillation pot. The centrifugation step was repeated with the remaining 500 ml of solvent/broth mixture. The distillation pot containing the combined solvent+polymer was then placed into a heating mantle and connected to a distillation set up maintained at atmospheric pressure. The mantle was adjusted to heat the solvent mixture to 180-185° C. Initially during the first heating stage, the solvent boiled over and condensed into the receiving flask. During the second stage heating, the P3HP polymer remaining in the distillation pot melted and then was thermally degraded to produce acrylic acid vapor. In the third stage, acrylic acid vapor was distilled, condensed and collected in the receiving flask which also contained hydroquinone (>98%, Sigma Aldrich) to prevent polymerization of the acrylic acid. When the distillation pot was dry, the heating mantle was shut off and the acrylic acid liquid allowed to cool to room temperature. The purity of the acrylic acid was determined by GC-MS to be >99.5% with a measured biobased content of 97%. The lower % biobased content was due to the presence of the hydroquinone stabilizer.

### Example 7

### Generation of 6-Valerolactone from P5HV Biomass

This example describes a process for producing biobased δ-valerolactone from whole fermentation broth containing biomass with poly-5-hydroxyvalerate (P5HV).

Aqueous fermentation broth consisting of E. coli microbial biomass containing poly-5-hydroxyvalerate polymer is prepared from glucose using procedures described in International Patent Pub. WO2010/068953 to Metabolix. The PHA titer for the broth would be approximately 150 g P5HV/kg of broth. The fermentation broth is pH adjusted to 11.5 by the addition of 8.5 g of lime/kg broth (Ca(OH)₂, Sigma Aldrich). 800 g of the pH 11.5 adjusted broth is then added to a 2 liter centrifuge flask and combined with 700 ml of reagent grade 2-pentanone (Sigma Aldrich, 90% pure containing up to 10% MIBK) that is heated to 60-65° C. prior to mixing. The 2 liter centrifuge flask is placed in a water bath set at 60-65° C. for 1 hour and the contents homogenized using a mixer every 5-10 minutes. After 1 hour, the centrifuge flask is removed from the water bath, placed in a heated centrifuge and spun down at 2200 rpm for 2 minutes. After centrifugation, the top liquid layer containing the P5HV polymer and 2-pentanone is decanted into a glass 1 liter distillation pot. The distillation pot is then placed into a heating mantle and connected to a distillation set up. The mantle is then heated to 180-185° C. In the first stage, the solvents are boiled over @102° C. for the 2-pentanone and 117.5° C. for the MIBK and condense into a receiving flask. During the second stage heating, the P5HV polymer remaining in the distillation pot melts and then is thermally degraded to produce 5-hydroxyvalerate (5HV) vapor which is refluxed back into the pot. In the third stage, refluxing is stopped and a vacuum is slowly applied (700 mmHg) while the 5HV vapor is distilled, condensed and collected in the receiving flask. When the distillation pot is dry, the vacuum and heating mantle are shut off and the 5-hydroxyvalerate liquid allowed to cool to room temperature. Total yield of 5HV liquid recovered would be 75-80% (g 5HV/g P5HV×100%). The purity of the 5-hydroxyvalerate is expected to be >99.5% with a biobased content of 99-100%.

### Example 8

### Generation of Ultra High Purity, Biobased Sodium Oxybate

In this example, the preparation of a pharmaceutical composition for treating diseases such as narcolepsy, cataplexy, fibromyalgia etc. utilizing sodium oxybate (sodium-gamma-hydroxybutyrate) is described. Ultra high purity GBL (>99.82% GBL) as prepared in Examples 2 and 3 is slowly added to a solution of NaOH (25 mol in 2 L of water and 400 ml of ethanol) in a reaction vessel with mechanical stirring. The reaction vessel is then heated to reflux for approximately 1 hour. After reaction, ethanol is removed by distillation resulting in an aqueous solution containing approximately 70% sodium-GHB by weight. Sodium-GHB produced using this approach should have a biobased content of at least 99% when tested according to the standard ASTM-D6866-11 testing protocol. U.S. Pat. No. 8,263,650 describes a method of preparing a microbially stable sodium-GHB formulation by first dissolving sodium-GHB in DI water to a concentration of 500 mg/ml. The pH is then adjusted with malic acid, HCl, citric acid or other acids to a value from 7.3-8.5. These acids also act as buffers to maintain the pH within the optimum range to prevent conversion of the GHB to GBL and to prevent microbial growth during storage.

### Example 9

### Generation of Ultra High Purity, Biobased Deuterated Sodium Oxybate

Ultra high purity, biobased sodium-GHB having one or more hydrogen atoms replaced with deuterium atoms can be prepared by starting with biobased GBL or its salt as prepared in Examples 2, 3 and 8 following the procedure described in Patent Application No. US2012/0122952 assigned to Concert Pharmaceuticals. Ultra high purity, biobased GBL is first converted to its butyl ester by reaction with butanol using an acid catalyst. The butanol can be of petroleum or biobased origin. The GBL t-butyl ester is then reacted in deuterated methanol in the presence of potassium carbonate to effect a hydrogen-deuterium atom exchange. After the hydrogen-deuterium exchange is complete, the compound is saponified with sodium hydroxide to form the biobased deuterated sodium oxybate. Alternatively, deuterated GBL can be first prepared from P4HB by feeding deuterated sugars (such as glucose), acetic acid or other deuterated feedstocks during fermentation to produce the deuterated analog of poly-4-hydroxybutyrate polymer. The polymer is then process as outlined in Examples 1 and 2 to produce ultra-high purity deuterated GBL.

### Example 10

### Generation of Ultra High Purity Biobased Fluorinated Sodium Oxybate

International Patent Application No. WO2102/142162 outlines a method and materials for fluorinating hydroxyl organic compounds such as pharmaceutical intermediates or precursors. The method can be applied to the ultra-high purity biobased sodium oxybate as prepared in Example 8 to produce an ultra-high purity fluorinated sodium oxybate pharmaceutical composition.

### Example 11

### Generation of Immediate Release, Ultra High Purity, Biobased Sodium Oxybate Solid Dosage Formulation

Patent application US20110111027 assigned to Jazz Pharmaceuticals discloses a solid dosage form for sodium oxybate which when taken orally is capable of quickly releasing 90% of the gamma-hydroxybutyrate active pharmaceutical in less than 1 hour similar to the effect when administering liquid sodium oxybate. The formulation contains Na-GHB (70-90% by weight), a binder e.g. hydroxypropyl cellulose (1-10% by weight), a lubricant e.g. magnesium stearate (0.5-5% by weight) and a surfactant e.g. sodium lauryl sulfate (0.5-3% by weight). The ingredients can be combined either in a dry or wet granulation procedure and then pressed into a tablet. In the wet procedure ethanol was used to first dissolve the hydroxypropyl cellulose binder. Similar formulations could also be made by substituting the ultra high purity, biobased sodium oxybate prepared in Examples 8-10 into the immediate release formulation as described above. Thereby making an ultra high purity biobased, immediate release, sodium oxybate solid dosage tablet.

### Example 12

### Controlled Release Solid Dosage Forms of Biobased Ultra High Purity Sodium Oxybate

Patent application US20120076865 assigned to Jazz Pharmaceuticals discloses controlled release dosage forms for water soluble and hygroscopic drugs such as sodium oxybate. The formulation as disclosed includes both an immediate release coating of sodium oxybate and a controlled released solid core of sodium oxybate. The core is composed of Na-GHB (90-100% by weight) and a polymer binder such as hydroxypropylene cellulose or ethyl cellulose (1-10% by weight) that are used for preparing the solid tablets. Other components may be added to the controlled release core such as lubricants, surfactants, plasticizers, excipients, compression aids or other fillers. The core is formed by wet granulation, roller compaction or direct compression. Once the core is formed, it is then coated to facilitate the controlled release of the sodium oxybate in the GI tract as well as to retain the integrity of the unit dosage form. The coating is a blend of a polymer e.g. cellulose polymers (50-80% by weight), a pore former which modifies the permeability of the coating e.g. hydroxypropyl cellulose, sugars or organic acids and other fillers or additives. It is applied to the core at about 2.5-7.5% by weight of the total tablet weight. The thickness of the coating also imparts control of the rate of release of the sodium oxybate from the core and can be varied to modulate the delivery of the pharmceutical. The release profile sodium oxybate from the coated tablet was shown to be in the range of 6-8 hours or more. Prior to administering the coated tablet, it can also be coated with an immediate release film containing sodium oxybate as described in Example 11. In this way the tablet delivers a predetermined concentration of sodium oxybate within the first hour then maintains a sustained release profile over the next 6-8 hours. Similar controlled release formulations could be made by substituting the ultra high purity, biobased sodium oxybate prepared in Examples 8-10 into the formulation as described above. Thereby making an ultra high purity biobased, controlled release, sodium oxybate solid dosage tablet.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A process for the production of an ultra-pure, biobased gamma-butyrolactone product, comprising
a) combining a genetically engineered biomass comprising polyhydroxyalkanoate polymer, a solvent and optionally a catalyst;
b) mixing the biomass and solvent together while optionally applying heat;
c) separating the organic and aqueous phases of the biomass and solvent mixture;
d) removing the solvent from the biomass; and
e) converting the biomass comprising polyhydroxyalkanoate to a biobased chemical product; wherein the weight percent biobased chemical in the product is greater than 95% and wherein the product does not comprise acetamide, n-methyl pyrrolidone or n-ethyl pyrrolidone and fatty acids.

2. A process for the production of an ultra-pure, biobased product, comprising
a) combining a genetically engineered biomass comprising polyhydroxyalkanoate polymer, a solvent and optionally a catalyst;
b) mixing the biomass and solvent together while optionally applying heat;
c) separating the organic and aqueous phases of the biomass and solvent mixture;
d) removing the solvent from the biomass; and
e) converting the biomass comprising polyhydroxyalkanoate to a biobased chemical product; wherein the weight percent biobased chemical in the product is greater than 95% wherein the product is a biobased gamma-butyrolactone product when the biomass comprises a poly-4-hydroxybutyrate, a biobased crotonic acid product when the biomass comprises a poly-3-hydroxybutyrate, a biobased acrylic acid product when the biomass comprises a poly-3-hydroxypropionate, or a biobased delta-valerolactone when the biomass comprises a poly-5-hydroxyvalerate and wherein the product does not comprise acetamide, n-methyl pyrrolidone or n-ethyl pyrrolidone and fatty acids.

3. The process of claim 1 or 2, wherein step b) is a continuous operation.

4. The process of claim 1 or 2, wherein step c) is a continuous operation.

5. The process of claim 1 or 2, wherein step d) further comprises removing the solvent by heating the organic phase containing solvent and polyhydroxyalkanoate under atmospheric or vacuum distillation conditions.

6. The process of any one of claims 1-5, wherein step e) further comprises converting the biomass by heating under vacuum or atmospheric distillation conditions, wherein the remaining polyhydroxyalkanoate is converted to a biobased chemical product.

7. The process of any one of claims 1-6 wherein the genetically engineered biomass is from a recombinant host having a poly-4-hydroxybutyrate pathway, wherein the host has an inhibiting mutation in its CoA-independent NAD-dependent succinic semialdehyde dehydrogenase gene or its CoA-independent NADP-dependent succinic semialdehyde dehydrogenase gene, or having the inhibiting mutations in both genes, and having stably incorporated one or more genes encoding one or more enzymes selected from a succinyl-CoA: coenzyme A transferase wherein the succinyl-CoA:coenzyme A transferase is able to convert succinate to succinyl-CoA, a succinate semialdehyde dehydrogenase wherein the succinate semialdehyde dehydrogenase is able to convert succinyl-CoA to succinic semialdehyde, a succinic semialdehyde reductase wherein the succinic semialdehyde reductase is able to convert succinic semialdehyde to 4-hydroxybutyrate, a CoA transferase wherein the CoA transferase is able to convert 4-hydroxybutyrate to 4-hydroxybutyryl-CoA, and a polyhydroxyalkanoate synthase wherein the polyhydroxyalkanoate synthase is able to polymerize 4-hydroxybutyryl-CoA to poly-4-hydroxybutyrate.

8. The process of any one of claim 1, claim 2, or claim 7 wherein the genetically engineered biomass is from a recombinant host having stably incorporated one or more genes encoding one or more enzymes selected from: a phosphoenolpyruvate carboxylase wherein the phosphoenolpyruvate carboxylase is able to convert phosphoenolpyruvate to oxaloacetate, an isocitrate lyase wherein the isocitrate lyase is able to convert isocitrate to glyoxalate, a malate synthase wherein the malate synthase is able to convert glyoxalate to malate and succinate, a succinate-CoA ligase (ADP-forming) wherein the succinate-CoA ligase (ADP-forming) is able to convert succinate to succinyl-CoA, an NADP-dependent glyceraldeyde-3-phosphate dehydrogenase wherein the NADP-dependent glyceraldeyde-3-phosphate dehydrogenase is able to convert glyceraldehyde 3-phosphate to 1,3-bisphosphoglycerate forming NADPH+H⁺, an NAD-dependent glyceraldeyde-3-phosphate dehydrogenase wherein the NAD-dependent glyceraldeyde-3-phosphate dehydrogenase is able to convert glyceraldehyde 3-phosphate to 1,3-bisphosphoglycerate forming NADH+H⁺, a butyrate kinase wherein the butyrate kinase is able to convert 4-hydroxybutyrate to 4-hydroxybutyryl-phosphate, a phosphotransbutyrylase wherein the phosphotransbutyrylase is able to convert 4-hydroxybutyryl-phosphate to 4-hydroxybutyryl-CoA; and optionally having a disruption in one or more genes selected from yneI, gabD, pykF, pykA, maeA and maeB.

9. The process of any one of claims 1-8, wherein the process further includes an initial step of culturing a recombinant host with a renewable feedstock to produce a biomass.

10. The process of claim 9, wherein a source of the renewable feedstock is selected from glucose, fructose, sucrose, arabinose, maltose, lactose, xylose, methanol. ethanol, 1,4-butanediol, fatty acids, glycerin, vegetable oils, or biomass derived synthesis gas or a combination thereof.

11. The process of any one of claims 1-10, wherein the biomass host is a bacteria, yeast, fungi, algae, cyanobacteria, or a mixture of any two or more thereof.

12. The process of claim 11, wherein the biomass host is bacteria.

13. The process of claim 12, wherein the bacteria is selected from Escherichia coli, Alcaligenes eutrophus (renamed as Ralstonia eutropha), Bacillus spp., Alcaligenes latus, Azotobacter, Aeromonas, Comamonas, Pseudomonads), Pseudomonas, Ralstonia, Klebsiella), Synechococcus sp PCC7002, Synechococcus sp. PCC 7942, Synechocystis sp. PCC 6803, Thermosynechococcus elongatus BP-I, Chlorobium tepidum, Chloroflexusauranticus, Chromatium tepidum and Chromatium vinosum Rhodospirillum rubrum, Rhodobacter capsulatus, and Rhodopseudomonas palustris.

14. The process of claim 11, wherein the recombinant host is algae.

15. The process of any one of claims 1-14, wherein the first heating is at a temperature from about 40° C. to about 170° C.

16. The process of any one of claims 1-15, wherein the second heating is at a temperature from about 60° C. to about 220° C.

17. The process of any one of claims 1-16, wherein the solvent is 2-butanone, 2-pentanone, 3-pentanone, methyl isoamyl ketone, 2-heptanone, cyclohexanone, acetone, chloroform, methylene chloride, gamma-butyrolactone or gamma-hydroxybutyrate.

18. The process of any one of claims 1-17, wherein the solvent is 2-pentanone containing up to 10% by weight methyl isobutyl ketone.

19. The process of any one of claims 1-18, wherein the vacuum pressure is at least 700 mmHg or 0 mmHg.

20. The process of any one of claims 1-19, further comprising recovering the gamma-butyrolactone product.

21. The process of any one of claims 1-20, wherein the biobased product comprises less than 0.1% by weight of side products.

22. The process of any one of claims 1-21, wherein the product is gamma-butyrolactone and is further processed to form one or more of the following: 1,4-butanediol (BDO), tetrahydrofuran (THF), N-methylpyrrolidone (NMP), N-ethylpyrrolidone (NEP), 2-pyrrolidinone, N-vinylpyrrolidone (NVP) and polyvinylpyrrolidone (PVP).

23. The process of any one of claim 1-7, wherein the genetically engineered biomass is from a recombinant host having a poly-4-hydroxybutyrate pathway, wherein the host has optionally an inhibiting mutation in its CoA-independent NAD-dependent succinic semialdehyde dehydrogenase gene or its CoA-independent NADP-dependent succinic semialdehyde dehydrogenase gene, or having inhibiting mutations in both genes, and having stably incorporated genes encoding the following enzymes: a succinyl-CoA:coenzyme A transferase wherein the succinyl-CoA:coenzyme A transferase is able to convert succinate to succinyl-CoA, a succinate semialdehyde dehydrogenase wherein the succinate semialdehyde dehydrogenase is able to convert succinyl-CoA to succinic semialdehyde, a succinic semialdehyde reductase wherein the succinic semialdehyde reductase is able to convert succinic semialdehyde to 4-hydroxybutyrate, a CoA transferase wherein the CoA transferase is able to convert 4-hydroxybutyrate to 4-hydroxybutyryl-CoA, and a polyhydroxyalkanoate synthase wherein the polyhydroxyalkanoate synthase is able to polymerize 4-hydroxybutyryl-CoA to poly-4-hydroxybutyrate.

24. The process of any one of claims 1-7, wherein the genetically engineered biomass is from a recombinant host having stably incorporated genes encoding the following enzymes: a phosphoenolpyruvate carboxylase wherein the phosphoenolpyruvate carboxylase is able to convert phosphoenolpyruvate to oxaloacetate, an isocitrate lyase wherein the isocitrate lyase is able to convert isocitrate to glyoxalate, a malate synthase wherein the malate synthase is able to convert glyoxalate to malate and succinate, a succinate-CoA ligase (ADP-forming) wherein the succinate-CoA ligase (ADP-forming) is able to convert succinate to succinyl-CoA, an NADP-dependent glyceraldeyde-3-phosphate dehydrogenase wherein the NADP-dependent glyceraldeyde-3-phosphate dehydrogenase is able to convert glyceraldehyde 3-phosphate to 1,3-bisphosphoglycerate forming NADPH+H⁺, an NAD-dependent glyceraldeyde-3-phosphate dehydrogenase wherein the NAD-dependent glyceraldeyde-3-phosphate dehydrogenase is able to convert glyceraldehyde 3-phosphate to 1,3-bisphosphoglycerate forming NADH+H⁺, a butyrate kinase wherein the butyrate kinase is able to convert 4-hydroxybutyrate to 4-hydroxybutyryl-phosphate, a phosphotransbutyrylase wherein the phosphotransbutyrylase is able to convert 4-hydroxybutyryl-phosphate to 4-hydroxybutyryl-CoA; and optionally having a disruption in one or more genes selected from yneI, gabD, pykF, pykA, maeA and maeB.

25. The process of any one of claims 1-7, wherein the genetically engineered biomass is from a recombinant host having a poly-4-hydroxybutyrate pathway, wherein the host has stably incorporated one or more genes encoding one or more enzymes selected from a succinyl-CoA:coenzyme A transferase wherein the succinyl-CoA:coenzyme A transferase is able to convert succinate to succinyl-CoA, a succinate semialdehyde dehydrogenase wherein the succinate semialdehyde dehydrogenase is able to convert succinyl-CoA to succinic semialdehyde, a succinic semialdehyde reductase wherein the succinic semialdehyde reductase is able to convert succinic semialdehyde to 4-hydroxybutyrate, a CoA transferase wherein the CoA transferase is able to convert 4-hydroxybutyrate to 4-hydroxybutyryl-CoA, and a polyhydroxyalkanoate synthase wherein the polyhydroxyalkanoate synthase is able to polymerize 4-hydroxybutyryl-CoA to poly-4-hydroxybutyrate.

26. The process of any one of claims 1-7, wherein the genetically engineered biomass is from a recombinant host having stably incorporated one or more genes encoding one or more enzymes selected from: a phosphoenolpyruvate carboxylase wherein the phosphoenolpyruvate carboxylase is able to convert phosphoenolpyruvate to oxaloacetate, an isocitrate lyase wherein the isocitrate lyase is able to convert isocitrate to glyoxalate, a malate synthase wherein the malate synthase is able to convert glyoxalate to malate and succinate, a succinate-CoA ligase (ADP-forming) wherein the succinate-CoA ligase (ADP-forming) is able to convert succinate to succinyl-CoA, an NADP-dependent glyceraldeyde-3-phosphate dehydrogenase wherein the NADP-dependent glyceraldeyde-3-phosphate dehydrogenase is able to convert glyceraldehyde 3-phosphate to 1,3-bisphosphoglycerate forming NADPH+H⁺, an NAD-dependent glyceraldeyde-3-phosphate dehydrogenase wherein the NAD-dependent glyceraldeyde-3-phosphate dehydrogenase is able to convert glyceraldehyde 3-phosphate to 1,3-bisphosphoglycerate forming NADH+H⁺, a butyrate kinase wherein the butyrate kinase is able to convert 4-hydroxybutyrate to 4-hydroxybutyryl-phosphate, a phosphotransbutyrylase wherein the phosphotransbutyrylase is able to convert 4-hydroxybutyryl-phosphate to 4-hydroxybutyryl-CoA; and optionally having a disruption in one or more genes selected from yneI, gabD, pykF, pykA, maeA and maeB.

27. The process of any one of claims 1-26, wherein the biobased content of the gamma-butyrolactone product is at least 99%.

28. The process of any one of claims 1-27, wherein the biobased content is 100%.

29. The process of any one of claims 1-28, wherein the weight percent biobased chemical in the product is greater than 96%.

30. The process of any one of claims 1-28, wherein the weight percent biobased chemical in the product is between 95% and 99.5% without any further processing of the product.

31. The process of any one of claims 1-28, wherein the weight percent biobased chemical in the product is between 97% and 99.5% without any further processing of the product.

32. The process of any one of claims 1-28, wherein the weight percent biobased chemical in the product is between 98% and 99.5% without any further processing of the product.

33. A biobased gamma-butyrolactone product produced by the process of any one of the preceding claims.

34. A biobased crotonic acid product produced by the process of any one of claims 2-19.

35. A biobased acrylic acid product produced by the process of any one of claims 2-19.

36. A biobased delta-valerolactone product produced by the process of any one of claims 2-19.

37. The product of claim 33, wherein the gamma-butyrolactone product comprises less than 0.05% by weight of side products.

38. The product of claim 34, wherein the crotonic acid product comprises less than 0.05% by weight of side products.

39. The product of claim 35, wherein the acrylic acid product comprises less than 0.05% by weight of side products.

40. The product of claim 36, wherein the delta-valerolactone product comprises less than 0.05% by weight of side products.

41. A poly-4-hydroxybutyrate biomass produced from renewable resources which is suitable as a feedstock for producing the gamma-butyrolactone product of claim 1, wherein the level of poly-4-hydroxybutyrate in the biomass is greater than 50% by weight of the biomass.

42. The process of any one of claims 1-32, wherein product yield is about 76% by weight or greater based on one gram of a gamma-butyrolactone in the product per gram of poly-4-hydroxybutyrate.

43. The product of claim 32, wherein the gamma-butyrolactone product comprises less than 0.1% by weight of side products wherein the side products do not comprise acetamide, n-methyl pyrrolidone or n-ethyl pyrrolidone.

44. The process of any one of claims 1-32 or 42, wherein the solvent is environmentally safe for human contact.

45. The process of any one of claims 1-32, 42 or 44, wherein the catalyst is sulfuric acid, phosphoric acid, hydrochloric acid, acetic acid, methane sulfonic acid, p-toluene sulphonic acid, trifluroacetic acid, zinc chloride, an ion exchange resin, potassium hydroxide, sodium hydroxide, calcium hydroxide or potassium carbonate.

46. The process of any one of claims 1-32, 42, 44, or 45, wherein the catalyst is added at least at 0.1% to at least 10% by weight of the polyhydroxyalkanoate to the genetically engineered biomass.

47. The gamma-hydroxybutyrate of any one of claims 33, 41 or 42, wherein the gamma-hydroxybutyrate is partially or wholly deuterated.

48. The gamma-hydroxybutyrate product of claim 1, 29, 33, 38 or 39, wherein the gamma-hydroxybutyrate is partially or wholly fluorinated.

49. A pharmaceutical composition comprising a sodium salt of gamma-hydroxybutyrate from anyone of claims 33, 41, 42, 47 or 48, and one or more pharmaceutically acceptable carriers.

50. The pharmaceutical composition of claim 49 comprising, a solid dosage tablet which releases 90% by weight of the sodium oxybate within one hour.

51. The pharmaceutical composition of claim 49 comprising, a solid dosage tablet which releases 99% by weight or more of the sodium oxybate over a time period of six to eight hours.

52. The pharmaceutical composition of claim 49 further comprising an outer coating which releases 90% by weight of the sodium oxybate in the outer coating in less than one hour.
